(19) United States
(12) Patent Application Publication
(10) Pub. No.: US 2024/0145045 A1
(43) Pub. Date: May 2, 2024

(54) INFORMATION PROCESSING APPARATUS, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Yasuyuki HOSONO, Kanagawa (JP); Nobuya KITAMURA, Kanagawa (JP); Kenji NAGAMIYA, Kanagawa (JP); Tomohide HIRAGAMI, Kanagawa (JP)

(21) Appl. No.: 18/540,698

(22) Filed: Dec. 14, 2023

Related U.S. Application Data
(63) Continuation of application No. PCT/JP2022/023316, filed on Jun. 9, 2022.

(30) Foreign Application Priority Data
Jul. 29, 2021 (JP) 2021-124688

(51) Int. Cl. G16H 10/20; G16H 10/60

(52) U.S. Cl. CPC G16H 10/20; G16H 10/60

(57) ABSTRACT

An information processing apparatus includes at least one processor. The processor acquires input information, which is information input to the information processing apparatus, and generates symptom information regarding a symptom of a user, which is capable of being entered in a medical questionnaire, based on input information satisfying a predetermined condition within the input information.

### CROSS-REFERENCE TO RELATED APPLICATIONS

This application is a continuation application of International Application No. PCT/JP2022/023316, filed Jun. 9, 2022, the disclosure of which is incorporated herein by reference in its entirety. Further, this application claims priority from Japanese Patent Application No. 2021-124688, filed on Jul. 29, 2021, the disclosure of which is incorporated herein by reference in its entirety.

### BACKGROUND

### 1. Technical Field

The disclosed technology relates to an information processing apparatus, an information processing method, and an information processing program.

### 2. Description of the Related Art

The following technologies are known as technologies for supporting medical inquiries performed for a patient in a medical institution such as a hospital. WO2017/183085A describes a medical inquiry support system that aims to output an appropriate medical questionnaire item from a large number of medical questionnaire items.

JP2003-44595A describes a health support system that receives transmission of data indicating a content of each user's daily meals, stores the received data in an individual management database for each user, evaluates the content of each user's meals based on data accumulated in the individual management database according to predetermined criteria, creates report information for the user using an evaluation result, and transmits the report information to a mobile information terminal used by the user.

### SUMMARY

In a medical institution such as a hospital, a medical inquiry is performed to inquire of a new patient a subjective symptom, a medical history, and the like. By performing the medical inquiry, it becomes possible to perform a prompt diagnosis. In recent years, electronization of a medical questionnaire has been under consideration in order to enhance efficiency of medical operations. In a case of using an electronic medical questionnaire, the patient inputs answers to the medical questionnaire items displayed on a screen of a terminal device owned by the patient or a terminal device provided by the hospital.

In the medical questionnaire, for example, it is assumed that an input of date and time of onset when the patient has noticed a symptom is required, but there is a probability that the patient may not have a clear awareness of the date and time of onset. In such a case, entering ambiguous information in the medical questionnaire may pose a risk of hindering an accurate diagnosis. In addition, for example, in a case of receiving medical care at a plurality of hospitals, there can be the inconvenience of inputting the same content into the medical questionnaire of each hospital.

The disclosed technology has been made in view of the above-described points, and an object thereof is to effectively support an input to an electronic medical questionnaire provided by a medical institution such as a hospital.

According to the disclosed technology, there is provided an information processing apparatus comprising at least one processor. The processor is configured to acquire input information, which is information input to the information processing apparatus, and generate symptom information regarding a symptom of a user, which is capable of being entered in a medical questionnaire, based on input information satisfying a predetermined condition within the input information.

The input information satisfying the condition may be information including a content related to a physical condition, a symptom, health, feeling, emotion, or a disease name of the user.

The processor may be configured to generate the symptom information based on biological information of the user at a point in time corresponding to a point in time when the input information satisfying the condition is input. The processor may be configured to acquire the biological information from a measurement device that measures the biological information, through communication with the measurement device.

The input information may include a text input to the information processing apparatus or a selection item selected by using the information processing apparatus. The input information may be information input on an application installed on the information processing apparatus. The input information may include a search keyword input to a search engine for searching for information on a network. The input information may include a content transmitted or dispatched to a specified or unspecified party. The input information may include information on a product or service that has been purchased or a product or service that has been attempted to be purchased, through information communication via a network.

The processor may be configured to store, in a storage medium, input date and time information indicating date and time when the input information is input, in association with the input information. The processor may be configured to transmit the symptom information to a predetermined transmission destination. The processor may be configured to enter, in the medical questionnaire, an answer to a medical questionnaire item on the medical questionnaire based on the symptom information. The processor may be configured to transmit a completed medical questionnaire to a predetermined transmission destination. The processor may be configured to enter personal information, which is to be entered in the medical questionnaire, in the medical questionnaire based on personal information of the user stored in advance in a storage medium.

According to the disclosed technology, there is provided an information processing method executed by at least one processor provided in an information processing apparatus, the information processing method comprising: causing the processor to execute processing of: acquiring input information, which is information input to the information processing apparatus; and generating symptom information regarding a symptom of a user, which is capable of being entered in a medical questionnaire, based on input information satisfying a predetermined condition within the input information.

According to the disclosed technology, there is provided an information processing program for causing at least one processor provided in an information processing apparatus to execute processing of: acquiring input information, which is information input to the information processing apparatus; and generating symptom information regarding a symptom of a user, which is capable of being entered in a medical questionnaire, based on input information satisfying a predetermined condition within the input information.

According to the disclosed technology, it is possible to effectively support an input to an electronic medical questionnaire provided by a medical institution such as a hospital.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a medical inquiry system according to an embodiment of the disclosed technology;

FIG. 2 is a diagram showing an example of a hardware configuration of an information processing apparatus according to the embodiment of the disclosed technology;

FIG. 3 is a functional block diagram showing an example of a functional configuration of the information processing apparatus according to the embodiment of the disclosed technology;

FIG. 4 is a diagram showing an example of input information and corresponding biological information according to the embodiment of the disclosed technology;

FIG. 5A is a diagram showing an example of symptom information according to the embodiment of the disclosed technology;

FIG. 5B is a diagram showing an example of the symptom information according to the embodiment of the disclosed technology;

FIG. 6 is a diagram showing an example of an unfilled medical questionnaire according to the embodiment of the disclosed technology;

FIG. 7 is a diagram showing an example of the completed medical questionnaire according to the embodiment of the disclosed technology;

FIG. 8 is a diagram showing an example of personal information according to the embodiment of the disclosed technology; and

FIG. 9 is a flowchart showing an example of a flow of processing performed by executing a support program 110 according to the embodiment of the disclosed technology.

### DETAILED DESCRIPTION

Hereinafter, an example of an embodiment of the disclosed technology will be described with reference to the drawings. It should be noted that the same or equivalent constituent elements and portions in the drawings are assigned by the same reference numerals, and overlapping descriptions will be omitted.

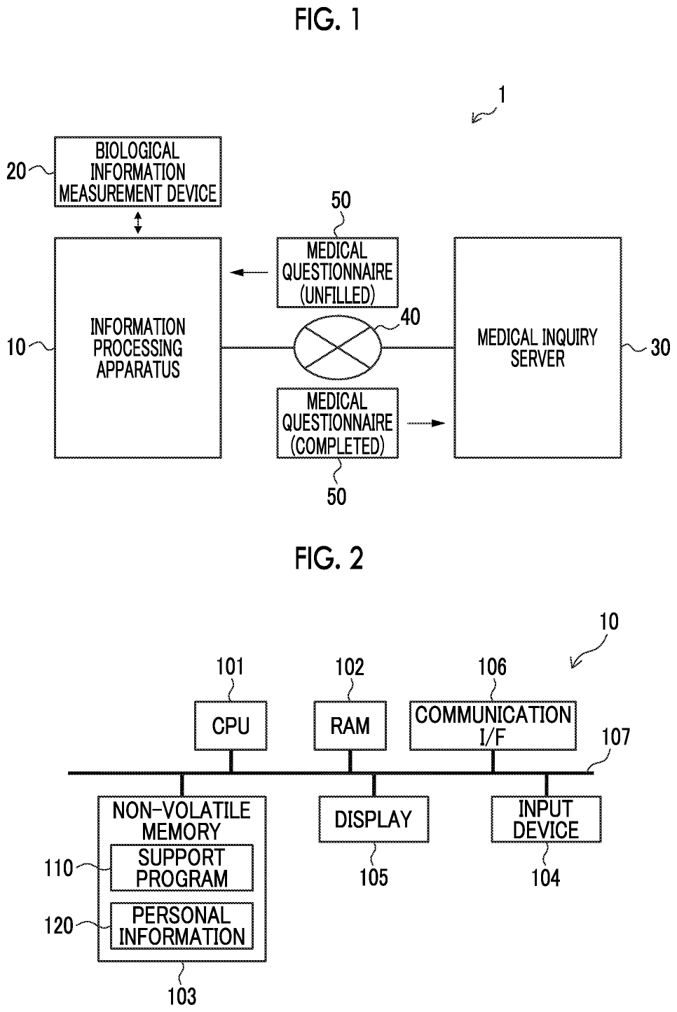

FIG. 1 is a diagram showing an example of a configuration of a medical inquiry system 1 according to the embodiment of the disclosed technology. The medical inquiry system 1 includes an information processing apparatus 10, a biological information measurement device 20, and a medical inquiry server 30.

The information processing apparatus 10 is a communication terminal apparatus used by a patient (hereinafter, referred to as a user) who attempts to receive medical care at a hospital. The information processing apparatus 10 may be, for example, a desktop computer, a laptop computer, a tablet computer, or a smartphone. The information processing apparatus 10 is connected to a network 40.

The biological information measurement device 20 measures biological information of the user. The biological information may be, for example, information indicating at least one of a body temperature, a heart rate, an electrocardiogram, an electromyogram, a blood pressure, an arterial blood oxygen saturation (SpO2), a blood glucose level, a lipid level, or the like. The biological information measurement device 20 may be a wearable device such as a smart watch provided with a sensor that measures these pieces of the biological information. The biological information measurement device 20 has a function of continuously measuring the biological information of the user in accordance with a predetermined time schedule and storing the biological information in an internal memory. The biological information measurement device 20 may measure the biological information for each predetermined period (for example, at intervals of 30 minutes). The biological information measurement device 20 is communicably connected to the information processing apparatus 10 and transmits the measured biological information to the information processing apparatus 10. It should be noted that the medical inquiry system 1 may comprise two or more biological information measurement devices that measure different types of the biological information.

The medical inquiry server 30 is a server managed and operated by a medical institution such as a hospital. The information processing apparatus 10 accesses the medical inquiry server 30 via the network 40 and downloads an unfilled medical questionnaire 50 provided by the medical inquiry server 30. The information processing apparatus 10 has a function of supporting the entering of the medical questionnaire 50 by the user and of transmitting the completed medical questionnaire 50 to the medical inquiry server 30.

FIG. 2 is a diagram showing an example of a hardware configuration of the information processing apparatus 10. The information processing apparatus 10 includes a central processing unit (CPU) 101, a random access memory (RAM) 102, a non-volatile memory 103, a display 105, an input device 104 including a keyboard, a mouse, and the like, and a communication interface 106. These pieces of hardware are connected to a bus 107.

The communication interface 106 is an interface used by the information processing apparatus 10 to perform communication with the biological information measurement device 20 and the medical inquiry server 30. A communication method may be any of wired or wireless communication. For wireless communication, for example, a method conforming to an existing wireless communication standard, such as Wi-Fi (registered trademark) or Bluetooth (registered trademark), can be applied. The non-volatile memory 103 is a storage device, such as a hard disk and a flash memory, and stores a support program 110 and personal information 120, which will be described below. The RAM 102 is a work memory used by the CPU 101 to execute processing. The CPU 101 loads the support program 110 stored in the non-volatile memory 103 into the RAM 102 and executes the processing in accordance with the support program 110. The CPU 101 is an example of a “processor” according to the disclosed technology. The non-volatile memory 103 is an example of a “storage medium” according to the disclosed technology.

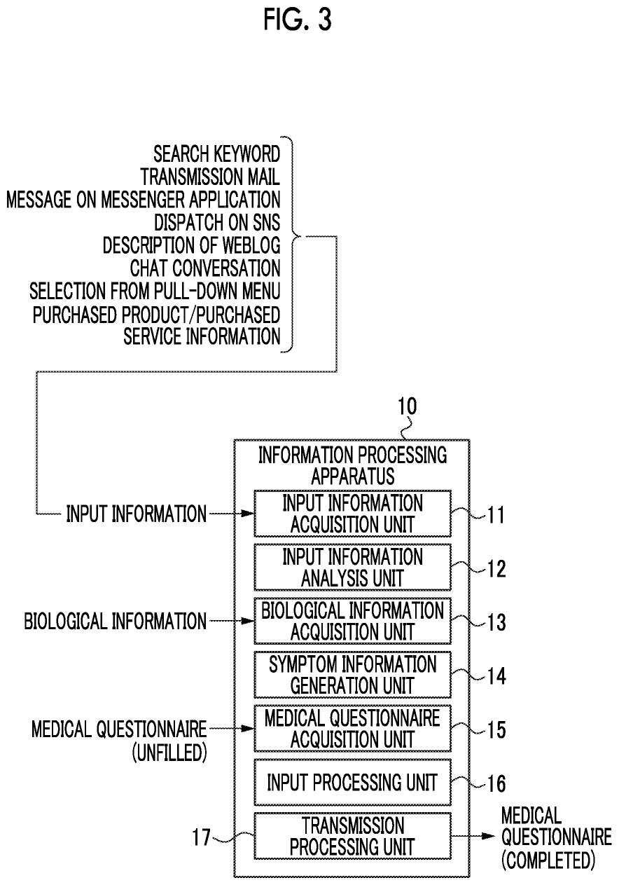

FIG. 3 is a functional block diagram showing an example of the functional configuration of the information processing apparatus 10. The information processing apparatus 10 includes an input information acquisition unit 11, an input information analysis unit 12, a biological information acquisition unit 13, a symptom information generation unit 14, a medical questionnaire acquisition unit 15, an input processing unit 16, and a transmission processing unit 17. The CPU 101 executes the support program 110, whereby the CPU 101 functions as the input information acquisition unit 11, the input information analysis unit 12, the biological information acquisition unit 13, the symptom information generation unit 14, the medical questionnaire acquisition unit 15, the input processing unit 16, and the transmission processing unit 17.

The input information acquisition unit 11 acquires input information input to the information processing apparatus 10. The input information can include a text input to the information processing apparatus 10 or a selection item selected by using the information processing apparatus 10. The input information may be information input on various applications installed on the information processing apparatus 10.

The text as the input information may be, for example, a search keyword input to a search engine for searching for information on the network. In addition, the text as the input information may be, for example, a text transmitted or dispatched to a specified or unspecified party, such as a transmission mail, a message on a messenger application, dispatch on the social networking service (SNS), description of the weblog, or a chat conversation. The selection item as the input information may be, for example, an item selected by using a graphical user interface (GUI) for performing selections from among a plurality of options, such as a pull-down menu or a radio button. Further, the input information may be information (for example, a product or service category, a product name, or a service name) regarding a product or service that has been purchased or a product or service that has been attempted to be purchased, from an electronic commerce (EC) site through information communication via the network using a web browser installed on the information processing apparatus 10. The input information acquisition unit 11 considers products or services viewed for the purchase of the product or service on the EC site or the like as the products or services that have been attempted to be purchased and acquires the information regarding these products or services as the input information. The input information acquisition unit 11 may acquire any combination of two or more of examples described above as the input information.

The input information acquisition unit 11 acquires the input information by collaborating with various applications such as a web browser, an email application, a messenger application, and an SNS application installed on the information processing apparatus 10. The input information acquisition unit 11 assigns input date and time information indicating date and time when the input information is input to the information processing apparatus 10 to the input information and stores the input information in the non-volatile memory 103.

The input information analysis unit 12 analyzes the input information acquired by the input information acquisition unit 11 and specifies input information satisfying a predetermined condition within the acquired input information. Specifically, the input information analysis unit 12 specifies the input information including a content related to a physical condition, a symptom, health, feeling, emotion, or a disease name of the user within the acquired input information as the input information satisfying the predetermined condition.

The input information analysis unit 12 may specify the input information satisfying the predetermined condition by determining whether or not a word registered in advance as the word related to the physical condition, the symptom, the health, the feeling, the emotion, or the disease name of the user is included in the text or the selection item as the input information. For example, in a case where the input information is a search keyword input to a search engine, the input information analysis unit 12 may specify the search keyword as the input information satisfying the predetermined condition in a case where the search keyword includes a word registered in advance such as “fever”, “cough”, “headache”, “abdominal pain”, “dizziness”, “cold”, “insomnia”, “anxiety”, “feeling down”, “fatigue”, “irritability”, “influenza”, “novel coronavirus”, or “COVID-19”. In addition, for example, in a case where the input information is a message input on the messenger application, the input information analysis unit 12 may determine the message as the input information satisfying the predetermined condition in a case where the message includes the registered word as in the examples described above. The word related to the physical condition, the symptom, the health, the feeling, the emotion, or the disease name of the user may include onomatopoeia, such as “throbbing”, “nauseous”, or “pitapat”.

In addition, in a case where the input information is information regarding a product that has been purchased or a product that has been attempted to be purchased through the information communication via the network, the input information analysis unit 12 may specify the input information including information on the product as the input information satisfying the predetermined condition in a case where the product is a medical supply, such as pharmaceuticals, or is related to a lifestyle of the user, such as alcohol and tobacco. Further, in a case where the input information is information regarding a service that has been purchased or a service that has been attempted to be purchased, through the information communication via the network, the input information analysis unit 12 may specify the input information including the information on the service as the input information satisfying the predetermined condition in a case where the service is related to health services or medical services aimed at maintaining, promoting, or recovering health, or alleviating, improving, or mitigating symptoms.

The biological information acquisition unit 13 acquires biological information of the user at a point in time corresponding to a point in time when the input information satisfying the predetermined condition is input. The “biological information at the point in time corresponding to the point in time when the input information is input” is biological information measured within a predetermined period before and after the point in time when the input information is input, and is, typically, biological information measured at a point in time closest to the point in time when the input information is input. The biological information acquisition unit 13 transmits, to the biological information measurement device 20, a transfer request of the biological information of the user at the point in time corresponding to the point in time when the input information satisfying the predetermined condition is input. The biological information acquisition unit 13 acquires the biological information transferred from the biological information measurement device 20 in response to the above-described transfer request. For example, in a case where the user inputs a text including a word of “fever”, the biological information acquisition unit 13 acquires, from the biological information measurement device 20, the biological information (for example, a body temperature) measured at a timing closest to the date and time when the text is input, through wired communication or wireless communication via the communication interface 106.

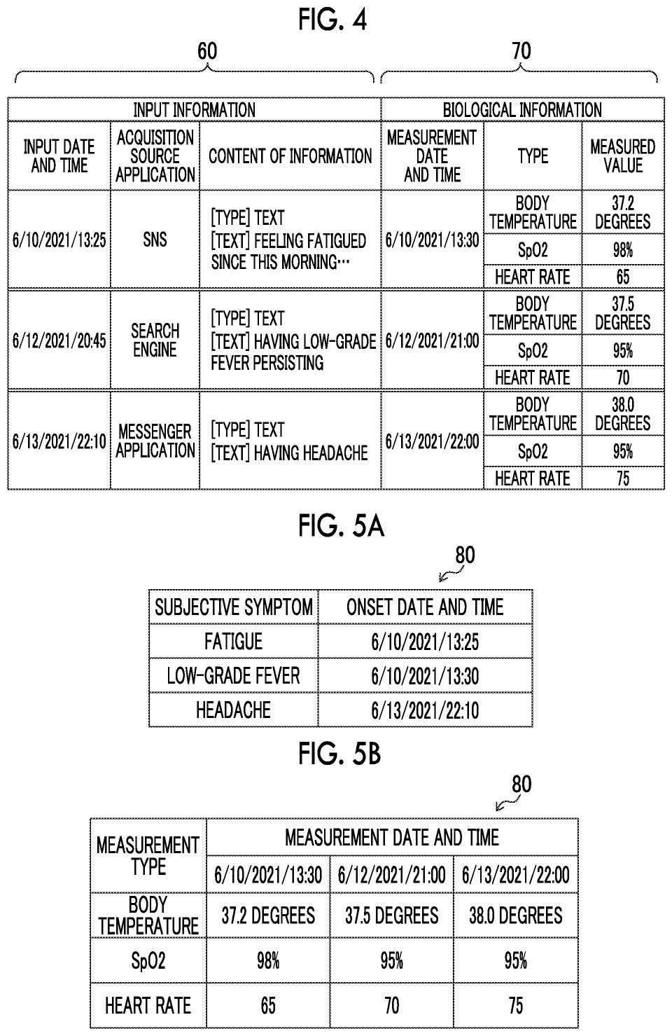

The biological information acquisition unit 13 stores the acquired biological information in the non-volatile memory 103 in association with the input information. FIG. 4 is a diagram showing an example of input information 60 and biological information 70 associated with each other. FIG. 4 illustrates a case where measured values of the body temperature, the SpO2, and the heart rate as the biological information are associated with each input information. As shown in FIG. 4, the input date and time information indicating the date and time when the input information is input, and a type of the application which is an acquisition source of the input information are assigned to the input information. In addition, as the content of the input information, a type of information (text/purchased product) and the content of the information (text content/product name) are recorded. Further, measurement date and time information indicating the date and time when the measurement is performed is assigned to each measured value as the biological information. The measurement date and time corresponds to the input date and time of the input information. FIG. 4 illustrates the following cases (1) to (3).

(1) The input information analysis unit 12 specifies a text including the word of “feeling fatigued” input on the SNS application installed on the information processing apparatus 10, as the input information satisfying the predetermined condition. The biological information acquisition unit 13 acquires, from the biological information measurement device 20, the measured values of the body temperature, the SpO2, and the heart rate as the biological information measured at a point in time (13:30 on Jun. 10, 2021) corresponding to a point in time when this text is input (13:25 on Jun. 10, 2021), and associates the above-described text with each measured value (the upper part of FIG. 4).

(2) The input information analysis unit 12 specifies a text including the search keyword of “having low-grade fever persisting” input to the search engine, as the input information satisfying the predetermined condition. The biological information acquisition unit 13 acquires, from the biological information measurement device 20, the measured values of the body temperature, the SpO2, and the heart rate as the biological information measured at a point in time (21:00 on Jun. 12, 2021) corresponding to a point in time when this text is input (20:45 on Jun. 12, 2021), and associates the above-described text with each measured value (the middle part of FIG. 4).

(3) The input information analysis unit 12 specifies a text including the word of “having a headache” input on the messenger application installed on the information processing apparatus 10, as the input information satisfying the predetermined condition. The biological information acquisition unit 13 acquires, from the biological information measurement device 20, the measured values of the body temperature, the SpO2, and the heart rate as the biological information measured at a point in time (22:00 on Jun. 13, 2021) corresponding to a point in time when this text is input (22:10 on Jun. 13, 2021), and associates the above-described text with each measured value (the lower part of FIG. 4).

The symptom information generation unit 14 estimates a symptom of the user based on the input information satisfying the predetermined condition and the biological information corresponding to the input information, which are stored in the non-volatile memory 103, and generates symptom information. The symptom information is information regarding a symptom of the user that can be entered in a medical questionnaire provided by a medical institution such as a hospital. The symptom information is not limited to the disease condition of the user and may include information regarding the feeling and the emotion of the user. FIGS. 5A and 5B are diagrams showing an example of symptom information 80 generated based on the input information 60 and the biological information 70 illustrated in FIG. 4.

As illustrated in FIG. 5A, the symptom information 80 includes a subjective symptom of the user and an onset date and time thereof. The symptom information generation unit 14 estimates that the user has noticed fatigue since 13:25 on Jun. 10, 2021, based on the word of “feeling fatigued” included in the text as the input information 60 illustrated in FIG. 4. In addition, the symptom information generation unit 14 estimates that the user has noticed low-grade fever since 13:30 on Jun. 10, 2021, based on the word of “having low-grade fever persisting” included in the text as the input information 60 and the measured value of the body temperature as the biological information 70, which are illustrated in FIG. 4. Further, the symptom information generation unit 14 estimates that the user has noticed a headache since 22:10 on Jun. 13, 2021, based on the word of “having a headache” included in the text as the input information 60 illustrated in FIG. 4. The symptom information generation unit 14 generates the symptom information 80 in accordance with the above-described estimation.

Further, as illustrated in FIG. 5B, the symptom information generation unit 14 generates the biological information associated with the input information as the symptom information 80. FIG. 5B illustrates the symptom information 80 including the measured values and the measurement dates and times of the body temperature, the SpO2, and the heart rate as the biological information acquired based on the input information. The symptom information generation unit 14 stores the generated symptom information 80 in the non-volatile memory 103.

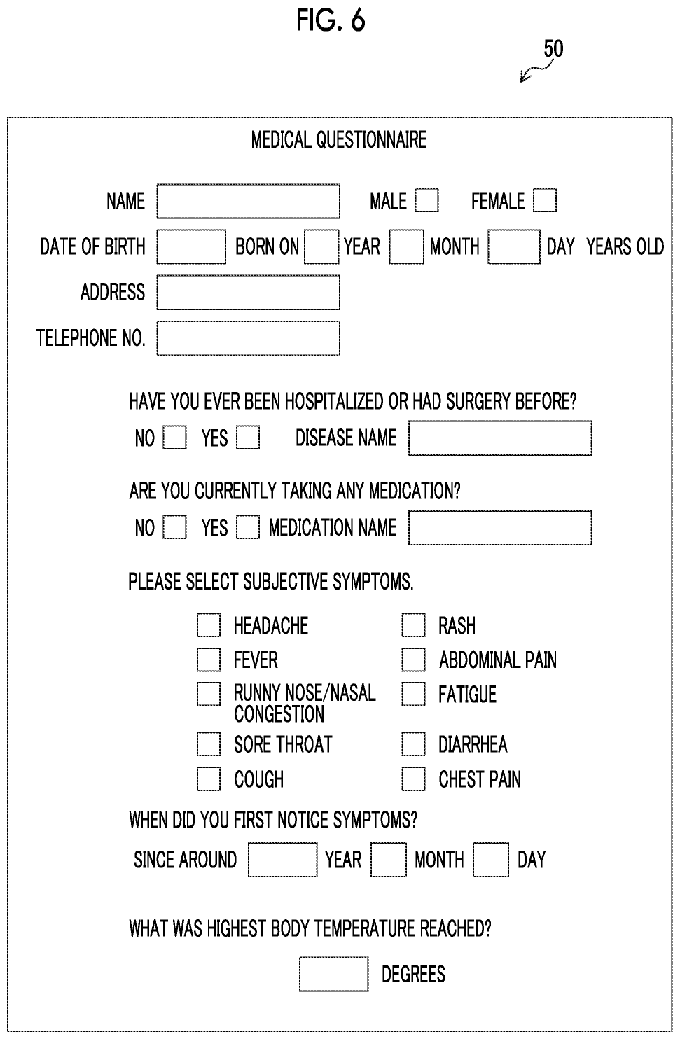

The medical questionnaire acquisition unit 15 acquires an electronic medical questionnaire provided by the medical inquiry server 30. The medical questionnaire is downloaded from the medical inquiry server 30 and is stored in the non-volatile memory 103. The medical questionnaire acquisition unit 15 acquires the medical questionnaire stored in the non-volatile memory 103. The medical questionnaire acquisition unit 15 may acquire the medical questionnaire directly from the medical inquiry server 30. FIG. 6 is a diagram showing an example of an unfilled medical questionnaire 50 acquired by the medical questionnaire acquisition unit 15.

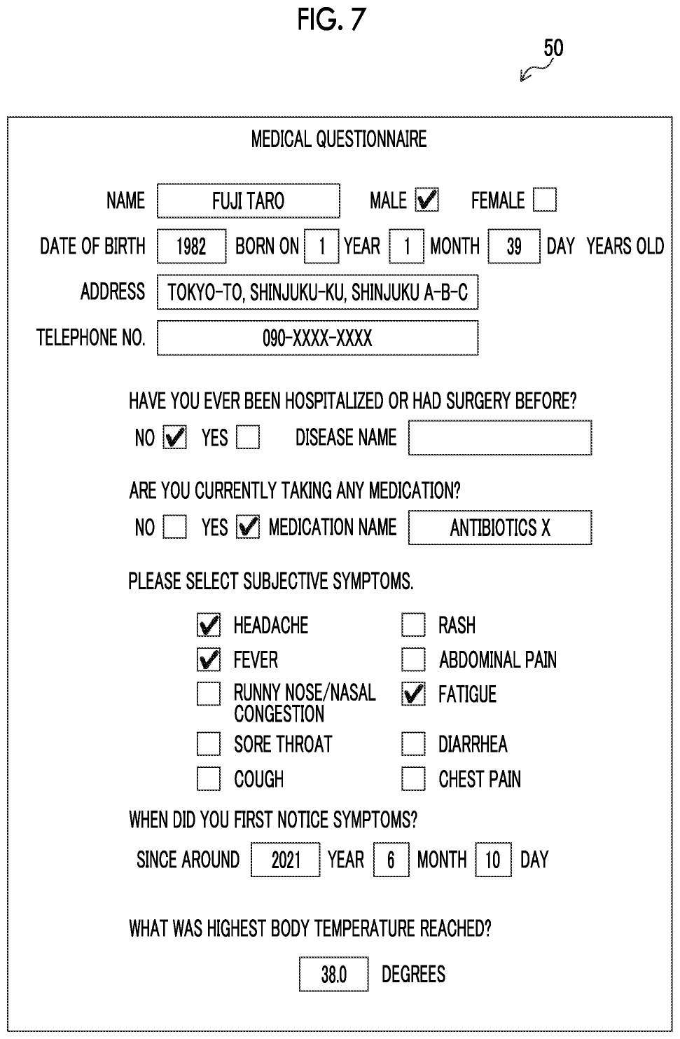

The input processing unit 16 enters an answer to the medical questionnaire item on the medical questionnaire acquired by the medical questionnaire acquisition unit 15, based on the symptom information generated by the symptom information generation unit 14. FIG. 7 is a diagram showing an example of the completed medical questionnaire 50 in which an answer to the medical questionnaire item is entered. The input processing unit 16 analyzes the medical questionnaire 50 and interprets the content of the medical questionnaire item.

The input processing unit 16, for example, based on the symptom information 80 illustrated in FIG. 5A, enters check marks in the checkboxes for “headache”, “fever”, and “fatigue” which are corresponding subjective symptoms for the medical questionnaire item “please select subjective symptoms” on the medical questionnaire 50. In addition, the input processing unit 16 enters “2021”, “6”, and “10” based on the symptom information 80 illustrated in FIG. 5A for the medical questionnaire item “when did you first notice the symptoms?” on the medical questionnaire 50. Further, the input processing unit 16 enters “38.0” based on the symptom information 80 illustrated in FIG. 5B for the medical questionnaire item “what was the highest body temperature reached?) on the medical questionnaire 50.

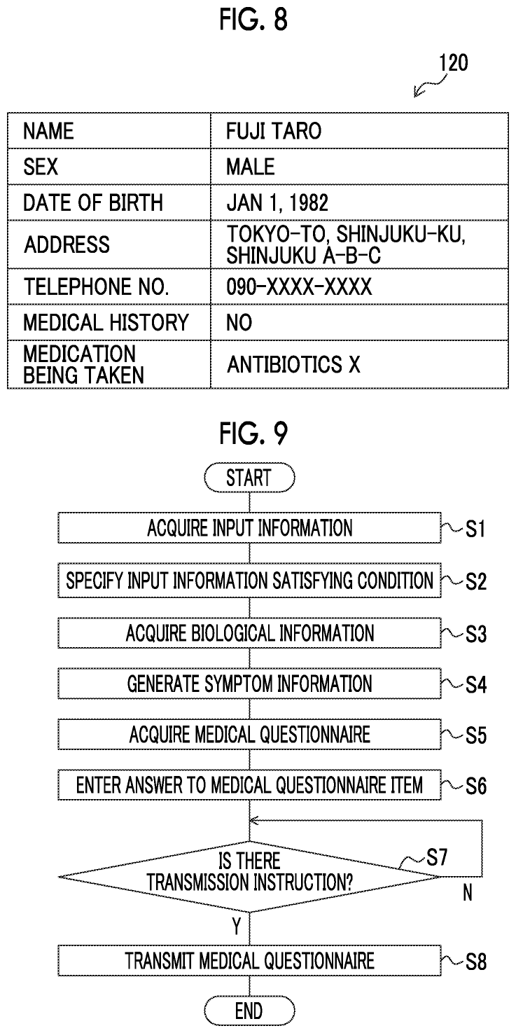

In addition, the input processing unit 16 enters personal information of the user, which is to be entered in the medical questionnaire 50, based on the personal information 120 stored in the non-volatile memory 103. FIG. 8 is a diagram showing an example of the personal information 120 stored in the non-volatile memory 103. The personal information 120 includes, for example, a name, a sex, a date of birth, an address, a telephone number, a medical history, a name of a medication being taken, and the like of the user. The personal information 120 is created by the user and is stored in the non-volatile memory 103. The input processing unit 16 enters the personal information (the name, the sex, the date of birth, the age, the address, the telephone number, the medical history, a name of the medication being taken) of the user, which is to be entered in the medical questionnaire 50, based on the personal information illustrated in FIG. 8.

The input processing unit 16 may enter an answer to the medical questionnaire item based on the information on the purchased product as the input information. For example, in a case where the medical questionnaire item includes an item related to the medication being taken or an item related to habits of alcohol consumption or smoking, the input processing unit 16 may enter an answer to the medical questionnaire item based on the input information including information on the product purchased through the information communication via the network. The medical questionnaire 50 in which the answer is entered by the input processing unit 16 is displayed on the display 105. The user can correct the answer entered by the input processing unit 16.

The transmission processing unit 17 transmits, to the medical inquiry server 30, the medical questionnaire 50 in which the answer to the medical questionnaire item is entered, based on a transmission instruction from the user.

FIG. 9 is a flowchart showing an example of a flow of processing performed by the CPU 101 through the execution of the support program 110. The support program 110 is executed, for example, in a case where the user issues an instruction to start the processing by operating the input device 104.

In step S1, the CPU 101 functions as the input information acquisition unit 11 to acquire the input information which is information input to the information processing apparatus 10. The input information acquisition unit 11 assigns the input date and time information to the acquired input information and stores the input information in the non-volatile memory 103.

In step S2, the CPU 101 functions as the input information analysis unit 12 to specify the input information satisfying the predetermined condition within the input information acquired in step S1. Specifically, the input information analysis unit 12 specifies the input information including a content related to the physical condition, the symptom, the health, the feeling, the emotion, or the disease name of the user as the input information satisfying the predetermined condition.

In step S3, the CPU 101 functions as the biological information acquisition unit 13 to acquire the biological information of the user at the point in time corresponding to the point in time when the input information satisfying the predetermined condition, which is specified in step S2, is input. The biological information acquisition unit 13 stores the acquired biological information in the non-volatile memory 103 in association with the input information.

In step S4, the CPU 101 functions as the symptom information generation unit 14 to generate the symptom information based on the input information satisfying the predetermined condition and the biological information corresponding to the input information.

In step S5, the CPU 101 functions as the medical questionnaire acquisition unit 15 to acquire the medical questionnaire downloaded from the medical inquiry server 30.

In step S6, the CPU 101 functions as the input processing unit 16 and enters the answer to the medical questionnaire item on the medical questionnaire acquired in step S5 based on the symptom information generated in step S4. The medical questionnaire in which the answer to the medical questionnaire item is entered is displayed on the display 105. The user can correct the answer entered by the input processing unit 16.

In step S7, the CPU 101 functions as the transmission processing unit 17 to determine whether or not there is a transmission instruction of the medical questionnaire in which the answer to the medical questionnaire item is entered. In a case where the user issues an instruction to transmit the medical questionnaire by operating the input device 104, in step S8, the transmission processing unit 17 transmits the medical questionnaire in which the answer to the medical questionnaire items is entered, to the medical inquiry server 30.

As described above, the information processing apparatus 10 according to the embodiment of the disclosed technology acquires the input information, which is information input to the information processing apparatus 10, and generates the symptom information regarding the symptom of the user, which can be entered in the medical questionnaire, based on the input information satisfying the predetermined condition within the input information. The input information satisfying the condition is information including the content related to the physical condition, the symptom, the health, the feeling, the emotion, or the disease name of the user. The input information includes a text input to the information processing apparatus 10 or a selection item selected by using the information processing apparatus 10. The input information can include information input on an application installed on the information processing apparatus.

With the information processing apparatus 10 according to the present embodiment, for example, the symptom information regarding the symptom of the user is generated based on the input information daily input by the user, such as the text transmitted or dispatched from the user by using various applications installed on the information processing apparatus 10, such as an email, a messenger application, and an SNS, and the search keyword, and the answer to the medical questionnaire item on the medical questionnaire is entered based on the symptom information. Therefore, even in a case where the user is not clearly aware of his/her symptom, it is possible to estimate the symptom of the user from the input information to generate the symptom information, and it is possible to enter accurate information in the medical questionnaire for the symptom of the user. In addition, it is possible to solve the problem of the inconvenience of inputting the same content into the medical questionnaire of each hospital in a case of receiving medical care at a plurality of hospitals. As described above, with the information processing apparatus 10 according to the present embodiment, it is possible to effectively support the input to the medical questionnaire.

In addition, with the information processing apparatus 10 according to the present embodiment, the symptom information is generated based on the biological information of the user as well as the input information, so that the effect of supporting the input to the medical questionnaire is further enhanced. In the present embodiment, a case where the symptom information is generated based on the input information and the biological information has been described as an example, but the symptom information may be generated without using the biological information.

The information processing apparatus 10 according to the present embodiment can be applied to a case of performing the entering of the medical questionnaire not only in a case where the user directly visits a hospital to receive the medical care but also in a case where the user at a remote place from the hospital receives online medical care.

In the present embodiment, an aspect has been described in which the information processing apparatus 10 enters the answer to the medical questionnaire item in the medical questionnaire and transmits the completed medical questionnaire to the medical inquiry server 30, but the disclosed technology is not limited to this aspect. The information processing apparatus 10 may transmit, to the medical inquiry server 30, the symptom information generated based on the input information and the biological information. In this case, the information processing apparatus 10 may appropriately perform processing of converting a data format of the symptom information into a proper format allowed in the medical inquiry server 30. In addition, in this case, the medical inquiry server 30 enters an answer to the medical questionnaire item on the medical questionnaire based on the symptom information transmitted from the information processing apparatus 10. Further, the information processing apparatus 10 may transmit both the completed medical questionnaire and the symptom information to the medical inquiry server 30.

In addition, in the present embodiment, a case where only the biological information of the user at the point in time corresponding to the point in time when the input information satisfying the predetermined condition is input is acquired from the biological information measurement device 20 has been described as an example, but the disclosed technology is not limited to this aspect. All the biological information measured by the biological information measurement device 20 may be acquired from the biological information measurement device 20. For example, the biological information acquisition unit 13 may acquire the biological information each time the measurement is performed by the biological information measurement device 20. In this case, the biological information acquisition unit 13 extracts the biological information corresponding to the point in time when the input information satisfying the predetermined condition is input within the acquired biological information, and generates the symptom information based on the extracted biological information.

In the above-described embodiment, for example, as a hardware structure of processing units that execute various types of processing, such as the input information acquisition unit 11, the input information analysis unit 12, the biological information acquisition unit 13, the symptom information generation unit 14, the medical questionnaire acquisition unit 15, the input processing unit 16, and the transmission processing unit 17, various processors to be described below can be used. The above-described various processors include, for example, a programmable logic device (PLD) which is a processor having a changeable circuit configuration after manufacturing, such as an FPGA, and a dedicated electrical circuit which is a processor having a dedicated circuit configuration designed to execute specific processing, such as an application specific integrated circuit (ASIC), in addition to the GPU and the CPU which is a general-purpose processor that executes software (programs) to function as various processing units, as described above.

One processing unit may be composed of one of these various processors or a combination of two or more processors of the same type or different types (for example, a combination of a plurality of FPGAs or a combination of a CPU and an FPGA). Alternatively, a plurality of processing units may be composed of one processor.

A first example in which a plurality of processing units are composed of one processor is an aspect in which one or more CPUs and software are combined to configure one processor and the processor functions as the plurality of processing units, as typified by a computer such as a client and a server. A second example is an aspect in which a processor that realizes functions of an entire system including a plurality of processing units with one integrated circuit (IC) chip is used, as typified by a system on chip (SoC) or the like. As described above, various processing units are composed of one or more of the above-described various processors as the hardware structure.

Further, as the hardware structure of these various processors, more specifically, an electrical circuit (circuitry) in which circuit elements, such as semiconductor elements, are combined can be used.

Moreover, in the above-described embodiment, an aspect has been described in which the support program 110 is stored (installed) in advance in the non-volatile memory 103, but the disclosed technology is not limited to this. The support program 110 may be provided in a form of being recorded on a recording medium, such as a compact disc read only memory (CD-ROM), a digital versatile disc read only memory (DVD-ROM), and a universal serial bus (USB) memory. Alternatively, a form may be employed in which the above-described support program 110 is downloaded from an external device via the network.

The disclosure of JP2021-124688 filed on Jul. 29, 2021 is incorporated in the present specification by reference in its entirety. Additionally, all documents, patent applications, and technical standards described in the present specification are incorporated in the present specification by reference to the same extent as in a case where the individual documents, patent applications, and technical standards have been specifically and individually stated to be incorporated by reference.

What is claimed is:

1. An information processing apparatus comprising:
at least one processor,
wherein the processor is configured to:
acquire input information, which is information input to the information processing apparatus; and
generate symptom information regarding a symptom of a user, which is capable of being entered in a medical questionnaire, based on input information satisfying a predetermined condition within the input information.

2. The information processing apparatus according to claim 1,
wherein the input information satisfying the condition is information including a content related to a physical condition, a symptom, health, feeling, emotion, or a disease name of the user.

3. The information processing apparatus according to claim 1,
wherein the processor is configured to generate the symptom information based on biological information of the user at a point in time corresponding to a point in time when the input information satisfying the condition is input, and the input information satisfying the condition.

4. The information processing apparatus according to claim 3,
wherein the processor is configured to acquire the biological information from a measurement device that measures the biological information, through communication with the measurement device.

5. The information processing apparatus according to claim 1,
wherein the input information includes a text input to the information processing apparatus or a selection item selected by using the information processing apparatus.

6. The information processing apparatus according to claim 1,
wherein the input information is information input on an application installed on the information processing apparatus.

7. The information processing apparatus according to claim 1,
wherein the input information includes a search keyword input to a search engine for searching for information on a network.

8. The information processing apparatus according to claim 1,
wherein the input information includes a content transmitted or dispatched to a specified or unspecified party.

9. The information processing apparatus according to claim 1,
wherein the input information includes information on a product or service that has been purchased or a product or service that has been attempted to be purchased, through information communication via a network.

10. The information processing apparatus according to claim 1,
wherein the processor is configured to:
store, in a storage medium, input date and time information indicating date and time when the input information is input, in association with the input information.

11. The information processing apparatus according to claim 1,
wherein the processor is configured to transmit the symptom information to a predetermined transmission destination.

12. The information processing apparatus according to claim 1,
wherein the processor is configured to enter, in the medical questionnaire, an answer to a medical questionnaire item on the medical questionnaire based on the symptom information.

13. The information processing apparatus according to claim 12,
wherein the processor is configured to transmit a completed medical questionnaire to a predetermined transmission destination.

14. The information processing apparatus according to claim 1,
wherein the processor is configured to enter personal information, which is to be entered in the medical questionnaire, in the medical questionnaire based on personal information of the user stored in advance in a storage medium.

15. An information processing method executed by at least one processor provided in an information processing apparatus, the information processing method comprising:
causing the processor to execute processing of:
acquiring input information, which is information input to the information processing apparatus; and
generating symptom information regarding a symptom of a user, which is capable of being entered in a medical questionnaire, based on input information satisfying a predetermined condition within the input information.

16. A non-transitory computer-readable storage medium storing an information processing program for causing at least one processor provided in an information processing apparatus to execute processing of:
acquiring input information, which is information input to the information processing apparatus; and
generating symptom information regarding a symptom of a user, which is capable of being entered in a medical questionnaire, based on input information satisfying a predetermined condition within the input information.
